(19) United States
(12) Patent Application Publication
(10) Pub. No.: US 2019/0143071 A1
(43) Pub. Date: May 16, 2019

(54) A BREATHING CIRCUIT

(71) Applicant: HAMAD MEDICAL CORPORATION, Doha (QA)

(72) Inventors: Yusuf Kassim ALREFAI, Doha (QA); Jafar HAMEED, Doha (QA)

(21) Appl. No.: 16/098,569

(22) PCT Filed: May 3, 2017
(86) PCT No.: PCT/GB2017/051235

§ 371 Date: Nov. 2, 2018

(30) Foreign Application Priority Data
May 3, 2016 (GB) 1607682.0

(51) Int. Cl. A61M 16/20; A61M 16/00; A61M 16/10; A61M 16/12; A61M 16/08

(52) U.S. Cl. CPC A61M 16/201; A61M 16/0078; A61M 16/104; A61M 16/0084; A61M 16/0833; A61M 16/0891; A61M 16/0875; A61M 16/12

(57) ABSTRACT

The present invention provides a breathing circuit for delivering a flow of gases to a patient, the circuit comprising a connector for connecting the circuit to a patient; an inspiration tube connected to the connector, the inspiration tube being connectable to a ventilator and for receiving an inflow of gases for the patient; and an expiration portion. The expiration portion comprises first and second expiratory tubes connected to the connector. The first expiratory tube is connectable to a ventilator, the first expiratory tube in use providing a first expiratory flow path from the patient via the connector and the first expiratory tube to the ventilator. The second expiratory tube is connected to an open-tail reservoir bag, the second expiratory tube in use providing a second expiratory flow path from the patient via the connector and the second expiratory tube to the reservoir bag. The system further comprises a valve operable to, in use, switch a flow of expiratory gases from the patient between the first and second expiratory flow paths. The system reduces storage space, material use and manufacturing costs whilst greatly enhancing convenience and safety for the patient.

### FIELD

THIS INVENTION relates to a respiratory or breathing circuit, more specifically to a paediatric anaesthesia circuit.

### BACKGROUND

Paediatric care often requires specialist carers and equipment, to deal with the wide variety of conditions that may affect babies, children and young people. In some cases, anaesthesia circuits intended for adult use are used in paediatric care. Sometimes, paediatric circuits are simply smaller than their adult equivalents, but additional features may also be included for added safety or convenience. Use of circuits intended for adults in paediatric care can be inconvenient in some cases, but ineffective or even dangerous in others.

An improved circuit system is sought to provide a cost-effective but more versatile and safer system suitable for paediatric anaesthesia care.

### PRIOR ART

FIGS. 1 and 2 illustrate prior art ‘T-piece’ and ‘circle’ breathing systems, respectively. The T-piece system 100 of FIG. 1 comprises an inspiratory tube 101, an angle piece connector 102 for attachment to a tracheal or intubation tube (not shown) and an expiratory tube 103 leading to an open-tail reservoir bag 104 for manual ventilation. The circle system 200 of FIG. 2 comprises an inspiratory tube 201 connected to an angle piece connector 202 for attachment to a tracheal or intubation tube and an expiratory tube 203.

The inspiratory tube 201 and expiratory tube 203 are suitable for connecting to a ventilator 110 (not shown) to aid respiration of the patient. The angle piece connector 202 connects to the patient via a mask or a tracheal or intubation tube (not shown). The circle system 200 may optionally further comprise a (closed) reservoir bag (not shown), e.g. for induction of anaesthesia.

Most ventilators/anaesthesia delivery units have at least three ports:
a. inspiratory (for a circle system);
b. expiratory (for a circle system); and
c. a main fresh gas flow outlet where a T-piece or coaxial system can be attached.

FIGS. 3a and 3b show the prior art circle system 200 connected to the inspiratory 110a and expiratory 110b ports on a ventilator 110 and the prior art T-piece system 100 connected to a fresh-gas-flow port 110c of the ventilator 110.

If the anaesthetist chooses to use the circle system only with its reservoir bag for induction of anaesthesia and then followed by automatic ventilation, then this is possible without using the T-piece system. Alternatively, the anaesthetist can use the T-piece system for induction followed by the circle system—here they have to ensure that they switch between the two breathing circuits appropriately. FIG. 3b shows the knobs/switches that allow movement from the circle system to the T-piece system and vice-versa. In the prior art system of FIGS. 3a and 3b, two knobs A and B function to switch between the systems. In some cases, these knobs comprise A) a flow parameter control switch, e.g. for adjusting parameters of the gases in the delivered flow (percentage oxygen, percentage anaesthetic gases, flow rate etc.); and B) a flow mode switch for switching between return (for use with the circle system) and non-return/fresh flow (for use with the T-piece system) flow modes.

‘Paediatric Anaesthesia for beginners’ (Dr. Tom Lawson, 2011), available online at: http://www.apagbi.org.uk/sites/default/files/images/APA%20Guide%20latest%20Version%2011_9_13.pdf sets out additional background to the present invention. Protection may be sought for features disclosed in the present application in combination with material known in the art, including this referenced material.

For paediatric care, the best systems are designed specifically for paediatric use, e.g. by being suitably sized, lightweight and with the T-piece having an appropriately-sized reservoir bag, e.g. half a litre.

### BRIEF SUMMARY OF THE INVENTION

The inventors have designed a paediatric anaesthesia circuit operable in multiple modes with a mechanism for safely switching therebetween. The claimed invention is beneficial because it provides a relatively simple, efficient system that reduces storage space, material use and manufacturing costs whilst greatly enhancing convenience for the anaesthetist and safety for the patient.

A first aspect of the present invention provides a breathing circuit for delivering a flow of gases to a patient, comprising:
a connector for connecting the circuit to a patient;
an inspiration tube connected to the connector, the inspiration tube being connectable to a ventilator and for receiving an inflow of gases for the patient; and
an expiration portion, the expiration portion comprising first and second expiratory tubes connected to the connector, wherein:
   the first expiratory tube is connectable to a ventilator, the first expiratory tube in use providing a first expiratory flow path from the patient via the connector and the first expiratory tube to the ventilator;
   the second expiratory tube is connected to an open-tail reservoir bag, the second expiratory tube in use providing a second expiratory flow path from the patient via the connector and the second expiratory tube to the reservoir bag; and
   the system further comprises a valve operable to, in use, switch a flow of expiratory gases from the patient between the first and second expiratory flow paths.

The present invention further provides a breathing circuit as claimed.

### BRIEF DESCRIPTION OF THE FIGURES

In order for the present invention to be more readily understood, prior art and preferable embodiments of the invention are described, by way of example only, with reference to the accompanying drawings, in which:

FIG. 1 shows a top-down view of a prior art T-piece system;

FIG. 2 shows a top-down view of a prior art circle system;

FIGS. 3a and 3b are perspective views of a prior art system with the prior art circuit and T-piece systems connected to a ventilator;

FIG. 4 shows a top-down view of a first embodiment of the present invention;

FIGS. 5a and 5b show top-down views of the first embodiment, shown schematically to be operating in a first operational mode;

FIGS. 6a and 6b show top-down views of the first embodiment, shown schematically to be operating in a second operational mode;

FIG. 7 shows a close-up perspective view of the first embodiment connected to a ventilator;

FIGS. 8a to 8d are schematics of a first valve system for the present invention;

FIGS. 9, 10a and 10b are perspective schematics of a second valve system for the present invention;

FIG. 11 is a schematic of a variant of the second valve system shown in FIGS. 9, 10a and 10b; and

FIGS. 12a to 12d are schematics of a third valve system for the present invention.

### DETAILED DESCRIPTION OF THE INVENTION

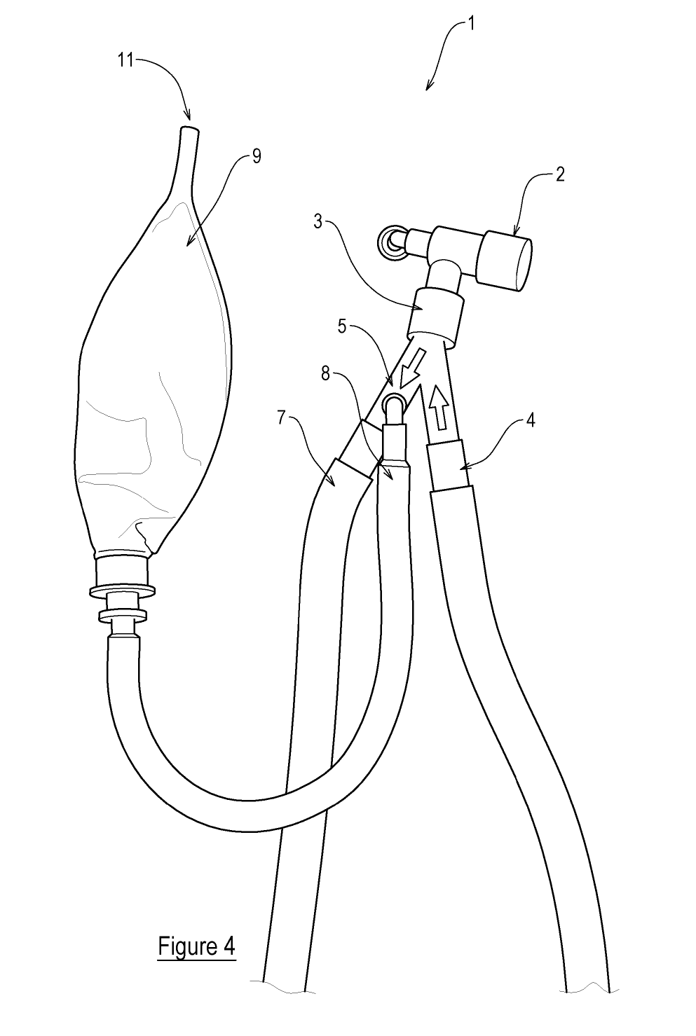

FIG. 4 illustrates a first embodiment of the present invention. The breathing circuit 1 of FIG. 4 comprises a Y-piece connector 3 for connecting the circuit to a patient, e.g. by way of an optional angle piece connector 2 and typically a mask (not shown) or a tracheal or intubation tube (not shown). An inspiration tube 4 is connected to the connector 3, the inspiration tube 4 suitable for receiving inflow from a ventilator 10 (not shown in FIG. 4) in use. The circuit 1 also comprises an expiration portion or limb 6, comprising first 7 and second 8 expiratory tubes, each connected to the connector 3. The first expiratory tube 7 is connectable to the ventilator 10 (not shown) in use and the second expiratory tube 8 is connected to a reservoir bag 9 having an open tail 11.

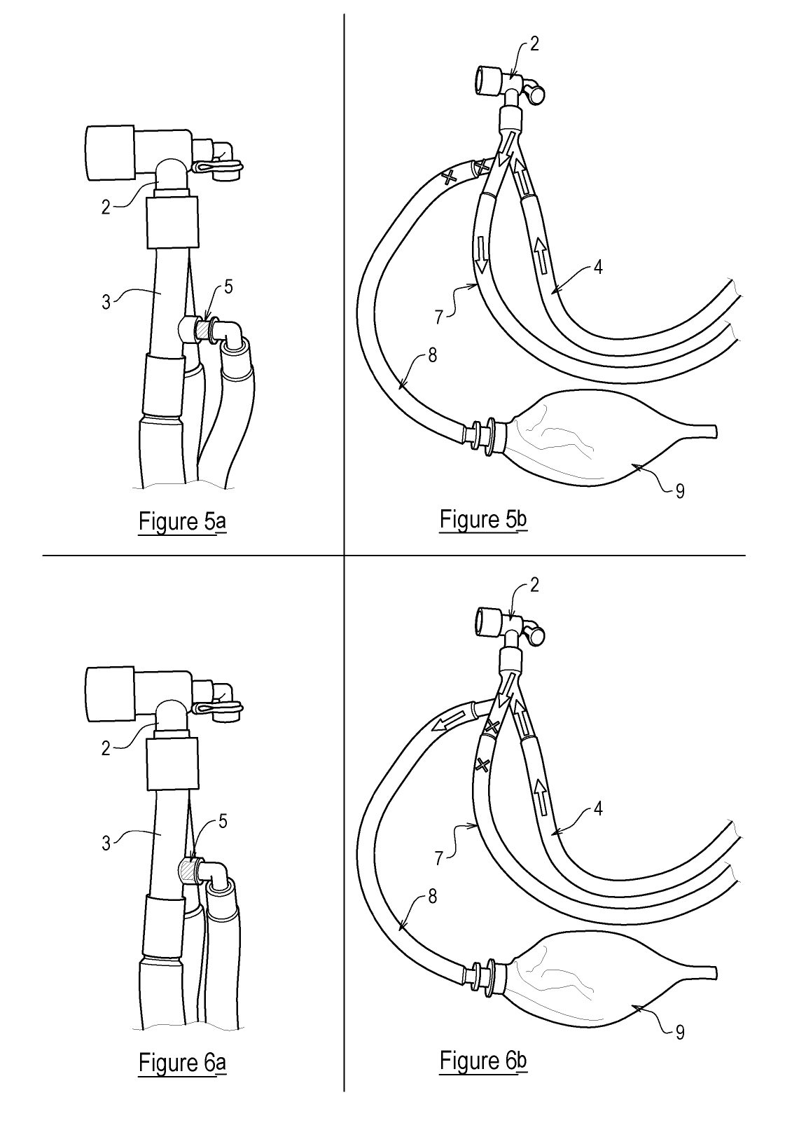

The system 1 further comprises a flow switch in the form of a valve 5 (better shown in FIGS. 5a and 5b), the valve 5 in use being operable to switch a flow of gases between the first and second expiratory tubes 7, 8 more specifically between:
a first flow path with inflow from ventilator 10 along the inspiratory tube 4 to the patient via the connector 3, including a first expiratory flow path from the patient through the first expiration tube 7 back to the ventilator 10; and
a second flow path with inflow (e.g. from ventilator 10 or a fresh gas flow outlet) along the inspiratory tube 4 to the patient via the connector 3, including a second expiratory flow path from the patient to the reservoir bag 9 via the second expiratory tube 8.

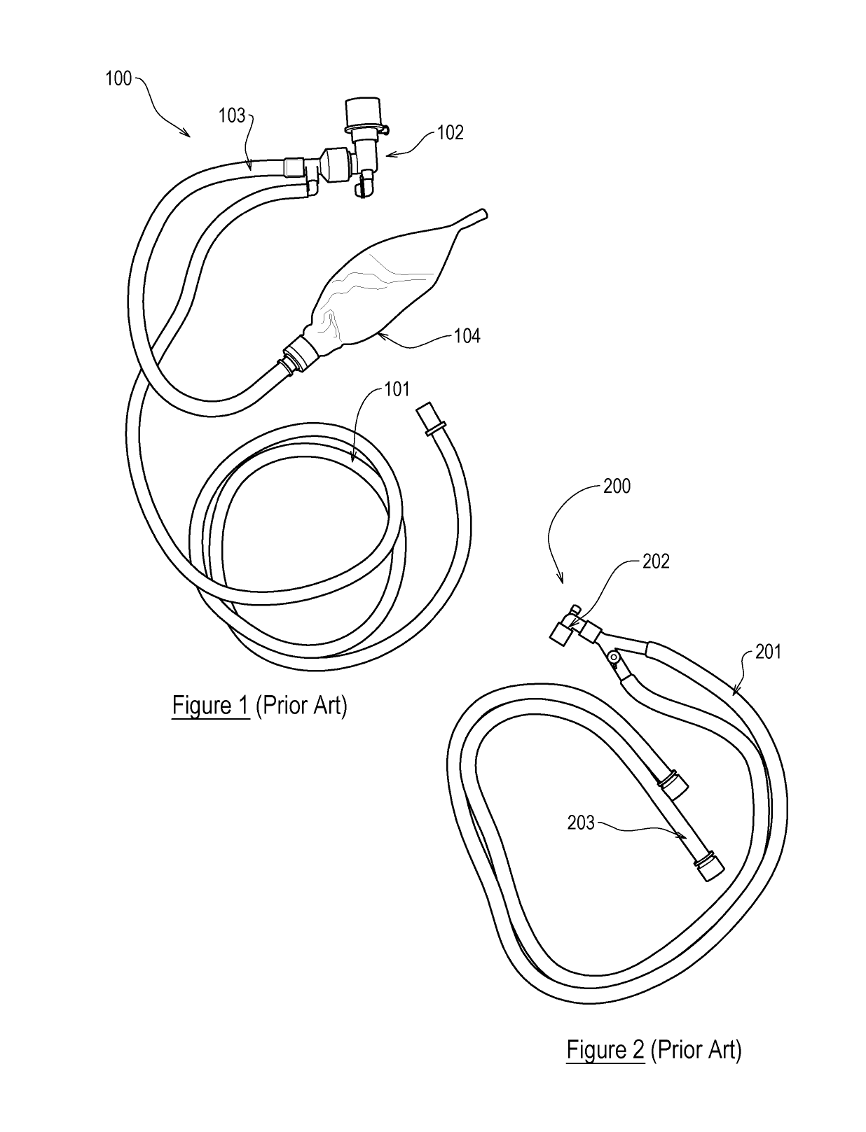

In essence, comparing the embodiment of FIG. 4 with those of FIGS. 1 and 2, in FIG. 4 the inspiration tube 4, the connector 3 and the first expiratory tube 7 form a circle flow system, whilst the inspiration tube 4, the connector 3, the second expiratory tube 8 and the reservoir bag 9 form a T-piece flow system; and the system is switchable between the circle and T-piece flow systems. The system is connectable to the ventilator 10, which may be operable in return (for use with the circle system) and non-return/fresh flow (for use with the T-piece system) flow modes. In preferred embodiments, the same inspiratory inflow path from the ventilator 10 is used in both systems without requiring any mode or ventilator port switching.

The switch/valve 5 of the circuit 1 and its operation are now described in more detail. Preferred embodiments are described in relation to the breathing circuit 1, but protection may be sought for the switch/valve 5 in isolation.

One embodiment of the valve 5 is shown in detail in FIGS. 5a to 6b. In FIG. 5a, the valve 5 is in a first ‘valve up’ position, permitting flow along the first flow path (i.e. with inflow from the ventilator 10 along the inspiratory tube 4 to the patient via the connector 3 and expiratory flow along the first expiratory flow path), as explained above and illustrated in FIG. 5b. In FIG. 6a, the valve 5 is in a second ‘valve pushed in’ position, permitting flow along the second flow path, (i.e. with the same inflow path but with expiratory flow along the second expiratory flow path) as explained above and illustrated in FIG. 6b.

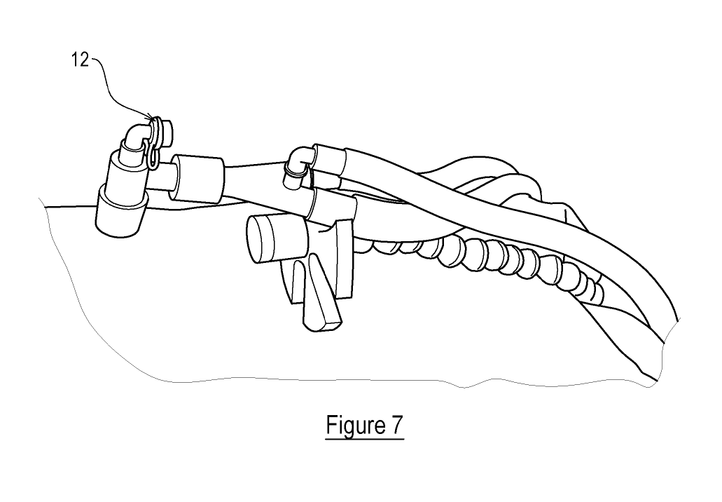

The circuit 1 is shown connected to a ventilator 10 in FIG. 7. FIGS. 5a-7 show the valve 5 at a juncture of the first and second expiratory flow tubes 7,8 which are connected together by the Y-piece connector 3. As shown best in FIG. 7, the optional angle piece connector 2 preferably comprises a CO₂ port 12 for capnography.

In some embodiments, the circle flow portion of the circuit 1 additionally comprises a closed reservoir bag at a T-junction along the first expiratory tube 8, wherein the first expiratory tube 8 passes flow back to the ventilator past the closed reservoir bag. One-way flow valves may also be provided to direct flow appropriately through the circuit 1.

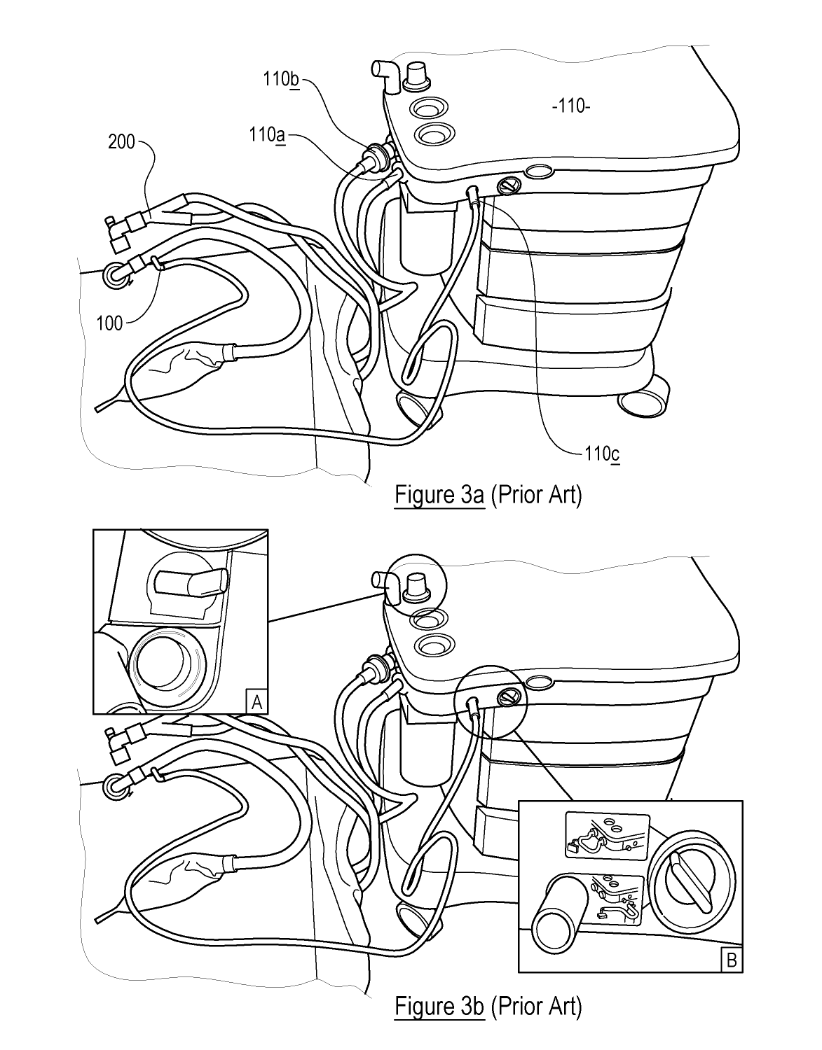

The system 1 of FIGS. 4-7 is beneficial, because it combines two breathing systems, avoiding the need to switch between two separate systems. The combined system provides faster, safer switching and reduces intervention. Comparing the present invention to the prior art arrangement of FIG. 3, in the present invention, no flow mode switching may be required at the ventilator (because the same inspiratory inflow path can used for both modes as described above with reference to FIG. 4) thus only flow parameter controls may be required on the ventilator with no mode switching controls needed—with reference to FIG. 3b, only knob A would be required in this kind of anaesthesia delivery unit instead of both knobs A and B in the prior art arrangement, hence also reducing possible errors.

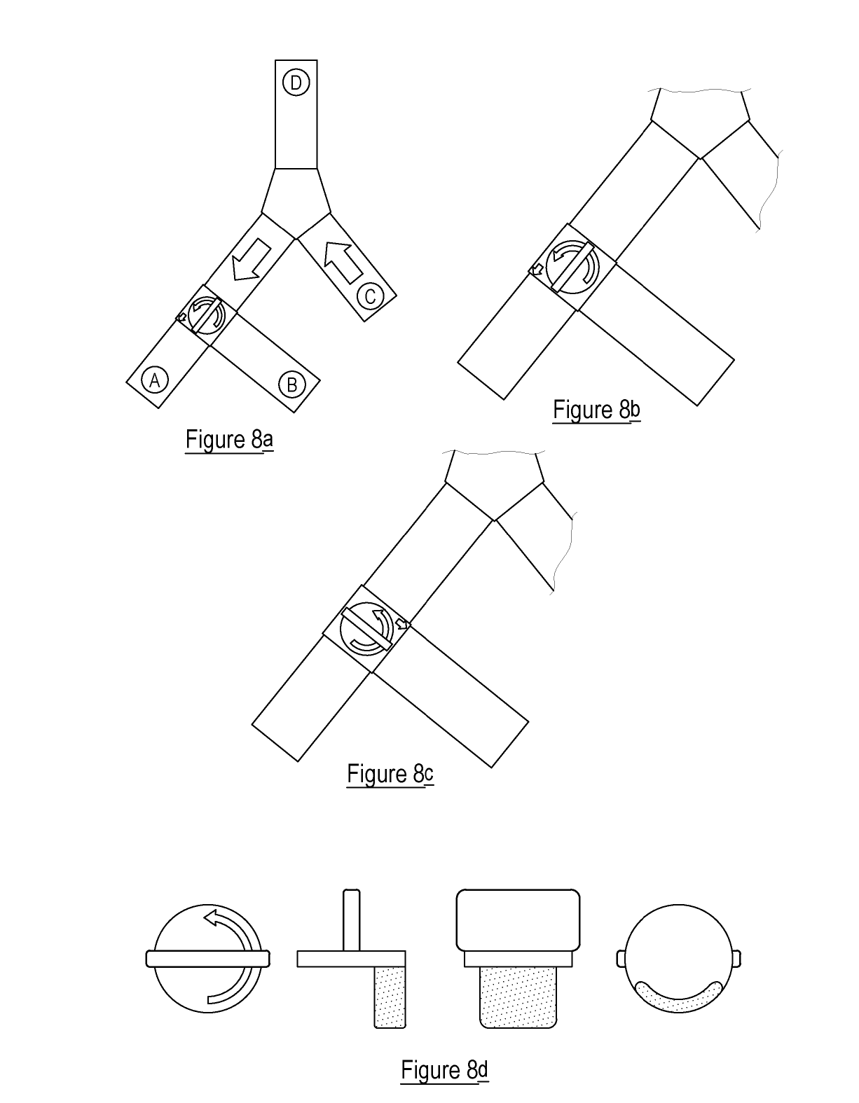

FIGS. 8a to 8d illustrate a first embodiment of the valve 5. FIG. 8a is a schematic representation of the tubes of the circuit 1 and shows the following:
A. expiration to ventilator (first expiratory flow path)
B. expiration to reservoir bag (manual resuscitator) (second expiratory flow path)
C. inspiration from ventilator (same for both first and second flow paths)
D. to patient via mask/intubation tube (same for both first and second flow paths)

FIG. 8b shows the valve 5 in the first position, permitting flow along the first flow path to/from the ventilator, with the return to the ventilator being via the first expiratory tube 7. The second expiratory flow path is blocked, with second expiratory tube 8 sealed. The seal can withstand pressures of up to 3 bar.

In this embodiment, the valve 5 is controlled by manual rotation and FIG. 8c shows the valve 5 in the second position, permitting flow along the second flow path, with expiration to the reservoir bag 9 via the second expiratory tube 8. The first expiratory flow path is blocked, with first expiratory tube 7 sealed. Again, the seal can withstand pressures of up to 3 bar.

In this embodiment, the valve profile is generally L-shaped. FIG. 8d shows above, side (left-right), side (top-bottom) and below views of a preferred switch valve profile for reliably sealing the flow paths.

The switch 5 is constructed from a rigid/hard material and the preferred diameter of the tubes that attach at points A, B, C and D are 10 mm for the tubes in the T-piece system and 15 mm for the tubes in the circuit system.

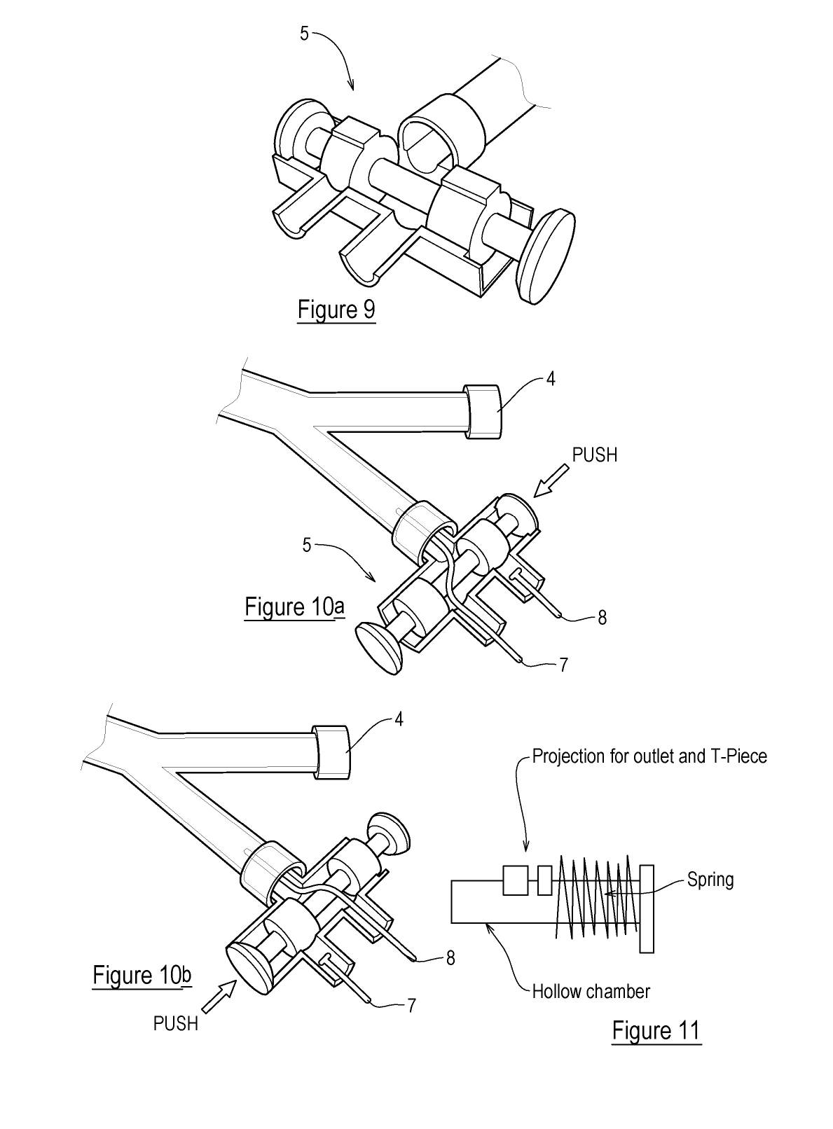

A preferred second embodiment of the valve 5 is illustrated in FIGS. 9, 10a and 10b. FIG. 9 shows a ‘plunger’ or ‘sliding shuttle’ valve which is moveable between at least two positions to select the expiratory flow path. Preferably, the valve 5 has plungers at each end for manually sliding the shuttle between the first and second positions.

FIG. 10a illustrates the valve 5 in the first position, with flow permitted through the leftmost flow path. FIG. 10b illustrates the valve 5 in the second position, with flow permitted through the rightmost flow path.

Preferably, the valve plungers are colour-coded to aid the operator in switching between circuits.

In some embodiments, the valve 5 is biased using a biasing element 13, e.g. a spring, preferably biased into one flow path/mode, so that the operator must keep the plunger depressed to permit flow into other flow paths/modes. A locking mechanism may be provided for securing the valve against the biasing element. In some embodiments, the biasing is to the first expiratory flow path which returns to the ventilator 10, since the reservoir bag 9 circuit is normally used for shorter periods. In other embodiments, the biasing is to the second expiratory flow path, using the T-piece system.

FIG. 11 shows a schematic of the shuttle valve 5 and illustrates one such embodiment where the valve 5 comprises a biasing element 13 in the form of a spring. The plunger is biased to the right, thus maintains the valve 5 in the flow mode of FIG. 10b.

In other embodiments, biasing elements such as springs are provided on both sides of the valve 5 to bias the valve 5 out of both T-piece and circle flow modes and into a third mode, which may be a ‘no-flow’ mode where both expiratory flow paths are closed and the operator must operate the valve 5 to use either other flow mode. This additional ‘no flow’ mode acts as a safety mechanism to ensure the operator selects a suitable flow mode.

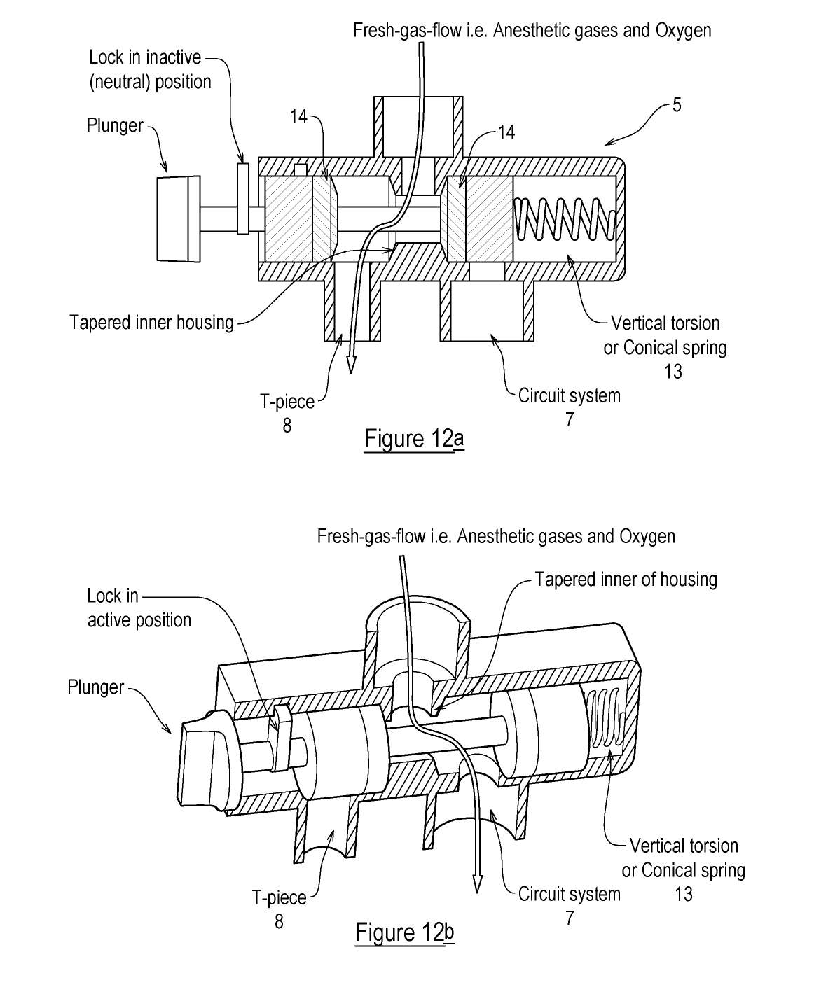

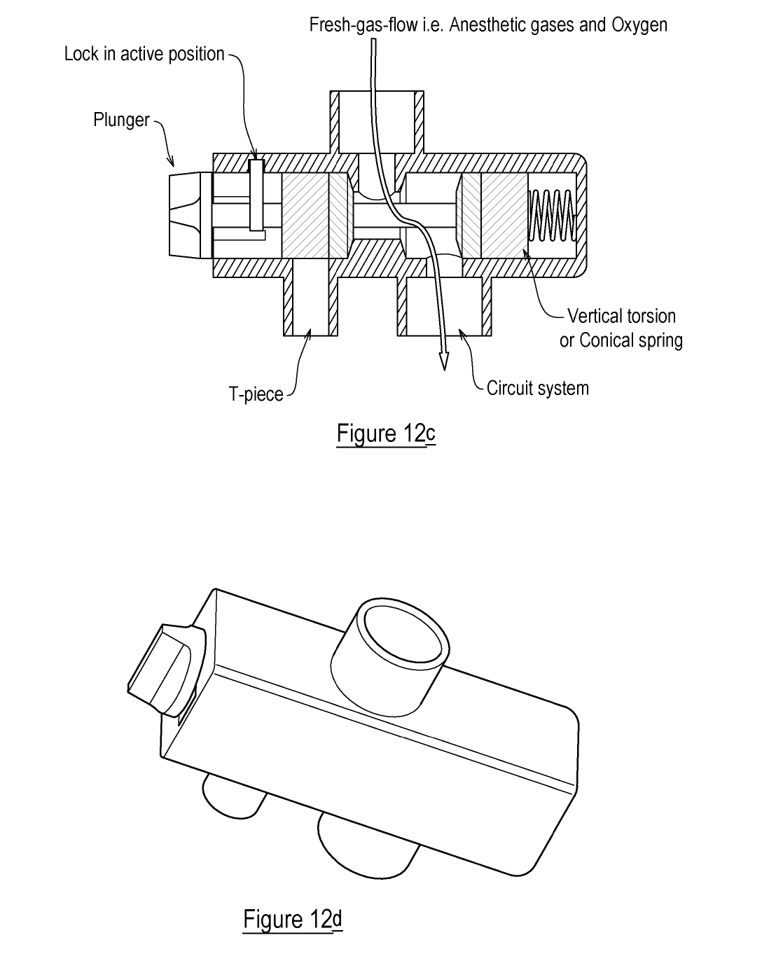

FIGS. 12a to 12d illustrate a third embodiment of the valve 5. In FIG. 12a, there is a plunger with a locking system and a valve biasing element 13 in the form of a spring (e.g. vertical torsion or conical) biased into a neutral (inactive) position, allowing fresh gas flow to the T-piece circuit, i.e. to the open-tail bag 9. In FIG. 12b, the plunger is pushed, turned and locked, allowing gas flow in the circle (circuit) system.

FIG. 12c illustrates all elements of the valve 5 and FIG. 12d shows the preferred external shape of the valve 5.

In the embodiment of FIGS. 12a to 12d, the valve 5 is biased into the neutral, unlocked position and also has a locked position, which secures the valve 5 in a position overcoming the biasing force. In this embodiment, the valve 5 permits flow only through the second expiratory flow path (to the T-piece system) in the neutral position and permits flow only through the first expiratory flow path (to the circle system) in the locked position. In other embodiments, the reverse arrangement is provided.

The locking mechanism comprises a notch or groove in an inner portion of a housing of the valve system and a tongue attached to the plunger, wherein the tongue is received in the notch, against the biasing force of the biasing element 13 to secure the valve 5 in the ‘active’ or locked position. In the embodiment shown, the tongue is rotated relative to the housing to engage the groove. O-rings 14 are provided to seal each expiratory flow path when not in use and the inner parts of the housing adjacent to the opening between the first and second expiratory tube 7,8 taper towards the opening, provide increased pressure at the periphery of the flow path, thus providing an improved seal. In this embodiment, the valve 5 is manually operated, but in other embodiments the valve may be electronically operated.

This third embodiment of the valve 5 is beneficial over the earlier embodiments because it is more compact, requires a single biasing element 13 in the form of a spring, and includes a locking mechanism to permit flow of anaesthetic gases and oxygen ONLY via the circle system. The valve 5 is also a single action switch rather than a shuttle valve, giving fewer points of possible failure.

In preferred embodiments, the circuit 1 further comprises a visual or audible indicator, such as a display or a light, for indicating a status of the circuit 1, e.g. OFF, STANDBY (system on but not in use) and the (expiratory) flow path (i.e. the flow mode e.g.: NO FLOW, RETURN/CIRCLE, FRESH FLOW/T-PIECE) in use. In further preferred embodiments, the circuit 1 further comprises a communications module for transmitting data preferably including the flow mode or flow parameters to other components and/or external devices (those not forming part of the breathing circuit itself), such as the ventilator 10.

Switching between separate T-piece and circle systems generally necessitates changes in the ventilator mode (from return flow to non-return/fresh flow and vice versa) and the present invention can eliminate the risk associated therewith by combining the two systems to avoid the need to switch the mode or automatically changing the mode of the circuit/ventilator where necessary, as appropriate. In further preferred embodiments, the circuit 1 is configured to switch a flow mode or parameter of the ventilator 10 and/or of the circuit 1 to coincide with the other of the ventilator 10 and the breathing circuit 1, to ensure that the two systems (the breathing circuit 1 and the ventilator 10) are configured to work together correctly. This is advantageous as it can prevent inappropriate use of the wrong circuit/wrong ventilator mode or unsuitable flow parameters.

For example, when the circuit is in the first flow mode to/from the ventilator, the circuit 1 communicates with the ventilator 10 and confirms that the ventilator 10 is functioning in the appropriate (return) flow mode with suitable flow parameters, else the system may be configured to switch a predetermined one of the circuit 1 and the ventilator 10 so that the modes coincide and/or the flow parameters are suitable, or to disable all flow and sound an alarm or display a message on a display. For example, ventilator or breathing circuit 1 may comprise a storage medium that stores acceptable or non-acceptable combinations of flow modes and/or flow parameters and the system checks the initial settings against the acceptable or non-acceptable combinations before making an adjustment or notifying the user.

The circuit 1 may therefore comprise a processor, sensor(s), motor(s), memory and/or a communications module configured to sense a position of the valve 5 and therefore determine the flow mode, and then communicate the mode to other components of the system or external devices such as the ventilator 10, preferably adjusting the flow mode of the circuit 1 in response to sensed information or data received from the external devices, or sending configuration information to adjust the component/external device for compatibility with the circuit 1. Particularly suitable sensors include temperature sensors, humidity sensors and flow rate sensors.

Preferably, the external device (ventilator 10) or the breathing circuit 1 can be configured as a MASTER device, with the other of the external device (ventilator 10) and the breathing circuit 1 thereby being configured as a SLAVE device and the slave device adjusts to function correctly with the master device.

In some embodiments, the circuit 1 comprises the ventilator 10, which may be an anaesthesia ventilator configured to deliver aesthetic gases to the patient.

Preferably, the ventilator comprises a communications module and the ventilator is configured to:
receive information from the circuit and adjust a mode or flow parameter of the ventilator for compatibility with the circuit; or
receive information from the circuit and send instructions back to the circuit to adjust a mode of the circuit for compatibility with the ventilator.

### INDEX TO REFERENCE NUMERALS

1. breathing circuit
2. angle piece connector
3. connector
4. inspiration tube or limb
5. valve
6. expiratory portion or limb
7. first expiratory tube or limb
8. second expiratory tube or limb
9. (open tail) reservoir bag
10. ventilator
11. open tail of reservoir bag
12. CO₂ port
13. valve biasing element
14. valve O-rings

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof. In particular, any specific element of one or more embodiments may be combined with any other embodiments disclosed herein.

1. A breathing circuit for delivering a flow of gases to a patient, comprising:
a connector for connecting the circuit to a patient;
an inspiration tube connected to the connector, the inspiration tube being connectable to a ventilator and for receiving an inflow of gases for the patient;
an open-tail reservoir bag; and
an expiration portion, the expiration portion comprising a first expiratory tube and a second expiratory tube connected to the connector, wherein:
the first expiratory tube is connectable to the ventilator, the first expiratory tube in use providing a first expiratory flow path from the patient via the connector and the first expiratory tube to the ventilator;
the second expiratory tube is connected to the open-tail reservoir bag, the second expiratory tube in use providing a second expiratory flow path from the patient via the connector and the second expiratory tube to the open-tail reservoir bag; and
the circuit further comprises a valve operable to, in use, switch a flow of expiratory gases from the patient between the first expiratory flow path and the second expiratory flow path.

2. The circuit of claim 1, wherein, in use:
the inspiration tube, the connector and the first expiratory tube form a circle flow system;
the inspiration tube, the connector, the second expiratory tube and the open-tail reservoir bag form a T-piece flow system; and
the circuit is switchable between the circle flow system and the T-piece flow system.

3. The circuit of claim 1, further comprising the ventilator, wherein the ventilator is switchable between a return mode and a non-return or fresh flow mode.

4. The circuit of claim 1, wherein the valve comprises a rotatable valve at a juncture of the first expiratory tube and the second expiratory tube.

5. The circuit of claim 1, wherein the valve comprises a plunger or a sliding shuttle at a juncture of the first expiratory tube and the second expiratory tube.

6. The circuit of claim 1, wherein the valve is biased into permitting flow via one of the first expiratory flow path or the second expiratory flow path.

7. The circuit of claim 4, wherein the valve is biased to prevent flow via either one or both of the first expiratory flow path and the second expiratory flow path.

8. The circuit of claim 5, wherein the valve comprises two opposed knobs for sliding the sliding shuttle between the first expiratory flow path and the second expiratory flow path.

9. The circuit of claim 1, wherein the valve is movable between a neutral, unlocked position and a locked position, wherein:
the valve is biased into the neutral, unlocked position and permits flow only through one of the first expiratory flow path or the second expiratory flow path in the neutral, unlocked position; and
the valve permits flow only through another one of the first expiratory flow path or the second expiratory flow path in the locked position.

10. The circuit of claim 9, wherein:
the valve permits flow only through the second expiratory flow path in the neutral, unlocked position; and
the valve permits flow only through the first expiratory flow path in the locked position.

11. The circuit of claim 1, further comprising at least one of a visual indicator or audible indicator, configured to indicate a status of the circuit.

12. The circuit of claim 11, wherein the visual indicator comprises a display or an illumination element configured to indicate the status of the circuit.

13. The circuit of claim 1, further comprising a communications module configured to at least one of:
send data relating to the circuit to at least one of other components of the circuit or external devices, or
receive data from at least one of other components of the circuit or external devices.

14. The circuit of claim 13, wherein the circuit is configured to at least one of:
switch between the first expiratory flow path the second expiratory flow path depending on at least one of:
the data sent to the at least one of other components of the circuit or external devices via the communications module, or
the data received from the at least one of other components of the circuit or external devices via the communications module; or
send instructions via the communications module to the at least one of other components of the circuit or external devices to adjust at least one of a mode or a parameter of the at least one of other components or external devices for compatibility with the circuit.

15. The circuit of claim 1, further comprising the ventilator, wherein the ventilator comprises a communications module and the ventilator is configured to at least one of:
receive data from the circuit and adjust at least one of a mode or a flow parameter of the ventilator for compatibility with the circuit; or
receive data from the circuit and send instructions to the circuit to adjust a mode of the circuit for compatibility with the ventilator.

16. The circuit of claim 3, wherein the ventilator is an anaesthesia delivery unit and is configured to provide anaesthetic gases flow.

17. The circuit of claim 1, further comprising at least one of an angle piece connector, a mask or an intubation tube.

18. The circuit of claim 1, wherein the circuit is an anaesthetic paediatric circuit.

19. The circuit of claim 6, wherein the valve is biased into permitting flow through the second expiratory flow path.

20. The circuit of claim 2, wherein the valve is movable between a neutral, unlocked position and a locked position, wherein:
the valve is biased into the neutral, unlocked position and permits flow only through one of the first expiratory flow path or the second expiratory flow path in the neutral, unlocked position; and
the valve permits flow only through another one of the first expiratory flow path or the second expiratory flow path in the locked position.
